(19) United States
(12) Patent Application Publication
(10) Pub. No.: US 2025/0161311 A1
(43) Pub. Date: May 22, 2025

(54) SMALL MOLECULE DIRECTED URINARY BLADDER TISSUE REGENERATION

(71) Applicants: Northwestern University, Evanston, IL (US); Ann and Robert H. Lurie Children's Hospital of Chicago, Chicago, IL (US)

(72) Inventors: Arun Sharma, Evanston, IL (US); Natalie J. Fuller, Evanston, IL (US); Matthew Bury, Chicago, IL (US)

(21) Appl. No.: 18/839,697

(22) PCT Filed: Feb. 17, 2023
(86) PCT No.: PCT/US2023/062846

§ 371 Date: Aug. 19, 2024

Related U.S. Application Data
(60) Provisional application No. 63/268,267, filed on Feb. 19, 2022.

(51) Int. Cl. A61K 31/52; A61P 13/10

(52) U.S. Cl. CPC A61K 31/52; A61P 13/10

(57) ABSTRACT

Disclosed are methods for regenerating bladder tissue. More specifically, disclosed are methods of regenerating bladder tissue by administering aryl hydrocarbon receptor effectors to a subject in need thereof.

### CROSS-REFERENCE TO RELATED APPLICATIONS

This application claims priority to, and the benefit of, U.S. Application No. 63/268,267 filed Feb. 19, 2022, the content of which is incorporated herein by reference in its entirety.

### BACKGROUND

Patients affected with severe and end-stage urinary bladder dysfunction secondary to radiation treatment for urological cancers, interstitial cystitis (IC), spina bifida (SB) and trauma have limited treatment options for bladder tissue replacement. Clinical manifestations of the pathologic bladder can include urinary incontinence, urinary tract infections, hydronephrosis, and eventual renal dysfunction/failure if left untreated or poorly managed. As bladder functionality becomes increasingly compromised, surgical intervention is required. Urinary diversion and bladder augmentation enterocystoplasty are the current gold standard surgical procedures to alleviate physiological problems of the pathological bladder. These procedures utilize autologous segments of bowel in an attempt to increase bladder compliance and capacity by physically enlarging the bladder. However, this procedure is accompanied by numerous short and long-term complications including infection, electrolyte imbalances, mucus and stone formation, spontaneous perforation, secondary and tertiary corrective surgeries, and the increased risk of malignancy. There are ˜14,000 bladder tissue replacement procedures performed in the United States annually that utilize intestinal segments. Using the IC as an example, it is estimated that ˜3-8 million women and ˜1-4 million men and an unknown number of children suffer from IC in the US from an unknown etiology resulting in millions of dollars spent annually treating disease with patients relying upon surgical intervention as a final option. Therefore, alternative options for increasing bladder compliance are desired.

### SUMMARY

Disclosed are methods of regenerating bladder tissue in a subject in need thereof. In an aspect, the method comprising administering to the subject a pharmaceutical composition comprising an effective amount of an aryl hydrocarbon receptor (AhR) effector for regenerating bladder tissue in the subject. In some embodiments, the AhR effector is a compound with the following formula or a pharmaceutically acceptable salt thereof:

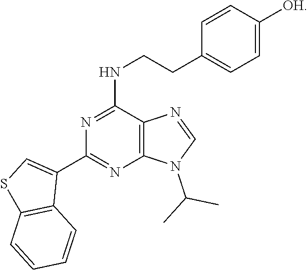 [SMILES: CC(C)n1cnc2c(NCCc3ccc(O)cc3)nc(-c3csc4ccccc34)nc21]

In some embodiments, administering comprises administering the effector to bladder tissue of the subject. In some embodiments, administering the AhR effector to bladder tissue of the subject comprises administering the compound coupled to a scaffold or released from an electronic actuator. In some embodiments, the scaffold comprises poly (1,8 octanediol-co-citrate) (POC). In some embodiments the regenerated bladder tissue comprises a ratio of about 1:1 muscle to collagen. In some embodiments, the method reduces one or more symptoms of bladder dysfunction. In some embodiments, the method results in increased vascularization and peripheral nerve innervation in the regenerated bladder tissue. In some embodiments, the method results in recovery of at least about 100% of the subject's pre-surgery bladder capacity.

Also provided herein are methods of promoting vascularization and peripheral nerve regeneration in surgically augmented urinary bladder tissue in a subject in need thereof. In an aspect, the method comprising administering to the subject a pharmaceutical composition comprising an effective amount of an aryl hydrocarbon receptor (AhR) effector to promote vascularization and peripheral nerve regeneration in the surgically augmented bladder tissue.

In some embodiments the AhR effector is a compound with the following formula or a pharmaceutically acceptable salt thereof:

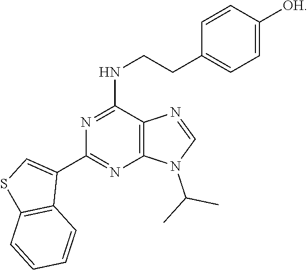 [SMILES: CC(C)n1cnc2c(NCCc3ccc(O)cc3)nc(-c3csc4ccccc34)nc21]

In some embodiments, administering comprises administering the AhR effector to bladder tissue of the subject. In some embodiments, administering the AhR effector to bladder tissue of the subject comprises administering the compound coupled to a scaffold or released from an electronic actuator. In some embodiments, the scaffold comprises poly (1,8 octanediol-co-citrate).

In some embodiments, the augmented bladder tissue comprises a ratio of about 1:1 muscle to collagen. In some embodiments, the method reduces one or more signs or symptoms of bladder dysfunction. In some embodiments, the method results in recovery of at least about 100% of the subject's pre-surgery bladder capacity.

Also provided herein is a method of increasing bladder capacity in a subject in need thereof. In an aspect, the method comprising administering to the subject a pharmaceutical composition comprising an effective amount of an aryl hydrocarbon receptor (AhR) effector to increase bladder capacity. In some embodiments, the subject has undergone surgical bladder augmentation.

In some embodiments, the AhR effector is a compound with the following formula or a pharmaceutically acceptable salt thereof:

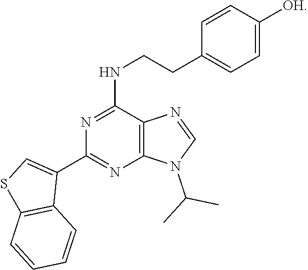 [SMILES: CC(C)n1cnc2c(NCCc3ccc(O)cc3)nc(-c3csc4ccccc34)nc21]

In some embodiments, administering comprises administering the AhR effector to bladder tissue of the subject. In some embodiments, administering the AhR effector to bladder tissue of the subject comprises administering the compound coupled to a scaffold or released from an electronic actuator. In some embodiments, the scaffold comprises poly (1,8 octanediol-co-citrate).

In some embodiments, the method reduces one or more signs or symptoms of bladder dysfunction.

Also provided herein is a composition comprising an aryl hydrocarbon receptor (AhR) effector coupled to a biodegradable elastomer. In some embodiments, the AhR effector is a compound with the following formula or a pharmaceutically acceptable salt thereof:

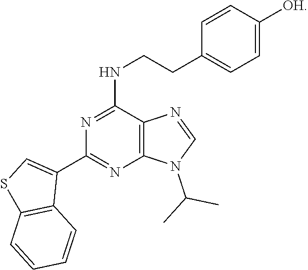 [SMILES: CC(C)n1cnc2c(NCCc3ccc(O)cc3)nc(-c3csc4ccccc34)nc21]

In some embodiments, the biodegradable elastomer comprises poly (1,8 octanediol-co-citrate).

Also provided herein are methods of inducing self-renewal and cellular differentiation of cells in a subject in need thereof. In an aspect, the method comprising administering to the subject a pharmaceutical composition comprising an effective amount of an aryl hydrocarbon receptor (AhR) effector for inducing self-renewal and cellular differentiation of cells. In some embodiments, the cells comprise epithelial cells or endothelial cells. In some embodiments, the AhR effector is a compound with the following formula or a pharmaceutically acceptable salt thereof:

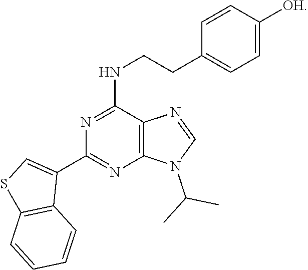 [SMILES: CC(C)n1cnc2c(NCCc3ccc(O)cc3)nc(-c3csc4ccccc34)nc21]

### BRIEF DESCRIPTION OF THE FIGURES

FIGS. 1A-1D. Expansion of primitive UCs. A. SR1 treatment increased the number of primitive nAhR+UCs (top panel; white arrows) that possessed capabilities of differentiating into mature cells (red arrows). B. 0 nM, 150 nM, 200 nM, and 250 nM SR1 treated UCs had peak numbers of nAhR+UCs (72.5±2.10; 159.75±8.44; 310.75±28.73; 342.75±20.97) cells at D7, respectively. Data represents means±SE. C. Reminiscent of primitive stem cells, nAhR+UCs maintained a high nucleus/cytoplasm ratio. SR1 treatment of nAhR+UCs tended to produce a modest increase in UCs with higher nucleus/cytoplasm ratios at D7, 200 nM. D. Overall cell size was increased in AhR−UCs while nAhR+UC maintained a small cell area.

FIGS. 2A-2C. Bladder smooth muscle regeneration and urothelium assessment. A. Trichrome staining of bladder tissue indicating a lack of muscle formation in the Saline group. B. Saline instilled animals (n=7) demonstrated 24.50±2.27% muscle content while SR1 instilled (n=7) and Cell-Seeded (n=4) animals were 47.00±1.81% and 50.72±1.67% muscle content at 10 weeks post-augmentation, respectively. SR1 vs Saline was p<0.0001 and Cell-Seeded vs Saline was p<0.0001. There was no statistical significance between SR1 and Cell-Seeded groups. C. Urothelium width (μm) was manually measured and quantified. Data demonstrate that Saline maintained a 40.82±2.15 μm width where SR1 and Cell-seeded constructs were 52.98±2.55 μm and 59.38±5.07 μm in width, respectively. Unmanipulated rats have an urothelium width ranging from 50-70 μm SR1 vs Saline p<0.05; Cell-Seeded vs Saline p<0.01. There was no statistical significance between SR1 and Cell-Seeded groups. p<0.05 was considered statistically significant. Data represents means±SE.

FIGS. 3A-3B. Bladder tissue vascularization. A. IF staining of bladder tissue for blood vessel markers. B. Saline instilled animals (n=7) demonstrated 1.490±0.13% vascularization while SR1 instilled (n=7) and Cell-Seeded (n=4) animals were 3.60±0.24% and 4.76±0.34% vascularization at 10 weeks post-augmentation, respectively. SR1 vs Saline was p<0.0001 and Cell-Seeded vs Saline was p<0.0001. There was a statistical significance between SR1 and Cell-Seeded groups, p<0.05. p<0.05 was considered statistically significant. Images are representative of multiple animals. Data represents means±SE.

FIGS. 4A-4B. Urodynamic Studies. A. Saline instilled animals demonstrated an approximate return to pre-augmentation intravesical pressure (40 cm H2O) at 10 weeks post-augmentation. SR1 instilled animals showed a decrease in pressure reaching ˜25 cm H2O. Similarly, Cell-Seeded animals returned to pre-augmentation values. B. With regards to percent bladder recovery, SR1 (n=7) vs Saline (n=7) was p<0.0001 (206.93±11.93% vs 80.28±5.61%) and Cell-Seeded (n=4) vs Saline was p<0.0001 (111.13±2.67% vs 80.28±5.61%). There was a statistical significance between SR1 and Cell-Seeded groups, p<0.0001. p<0.05 was considered statistically significant. Data represents means±SE and representative of multiple animals.

FIGS. 5A-5D. SR1 characterization. A. Cumulative release of SR1 that was coupled to POC scaffolds and measured in vitro. An ˜1.4× higher release was observed with 2 mM SR1 at the 30 day time point. B. Normal voiding/filling patterns and compliance of POC/SR1 augmented rats. C. An ˜1.8× increase in regenerated bladder muscle was observed in the POC/SRT group. D. With regards to regenerated bladder tissue vascularization, POC/SRT releasing scaffolds were higher at only 4 weeks post-augmentation. Data represents means±SE; n=2 per treatment group.

### DETAILED DESCRIPTION

The disclosed methods may be utilized to treat diseases and disorders associated with loss of bladder tissue and a need for regeneration of said tissue which may include, but are not limited to lack of bladder control, lack of bladder capacity, etc.

### Small Molecule Directed Urinary Bladder Tissue Regeneration

Disclosed herein are methods of using compounds and pharmaceutical compositions for regenerating bladder tissue in a subject in need thereof. As used herein, “a subject in need thereof” refers to a subject in need of regeneration of bladder tissue because of, for example, cystectomy due to radiation treatment for urological cancers, interstitial cystitis (IC), spina bifida (SB), or trauma. As used herein, “bladder cystectomy” refers to the full or partial surgical removal of the bladder. The inventors discovered that aryl hydrocarbon receptor (AhR) effectors, e.g., stemregenin 1 (SR1), can simultaneously stimulate primitive urothelial cell (UC) self-renewal and differentiation. As used herein, “aryl hydrocarbon receptor (AhR) effector” refers to a compound that interacts with, e.g., binds to, and specifically modulates the signaling of AhR. As used herein, “stemregenin 1” or “SR1” refers to an AhR effector compound with a formula:

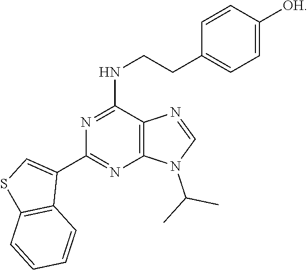 [SMILES: CC(C)n1cnc2c(NCCc3ccc(O)cc3)nc(-c3csc4ccccc34)nc21]

Accordingly, in a first aspect of the instant disclosure, methods for regenerating bladder tissue in a subject in need thereof are provided. In some embodiments, the methods comprise administering to the subject a pharmaceutical composition comprising an effective amount of an aryl hydrocarbon receptor (AhR) effector for regenerating bladder tissue in the subject. In some embodiments, the AhR effector is a compound with the following formula or a pharmaceutically acceptable salt thereof:

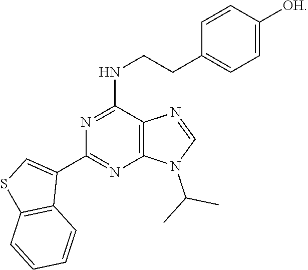 [SMILES: CC(C)n1cnc2c(NCCc3ccc(O)cc3)nc(-c3csc4ccccc34)nc21]

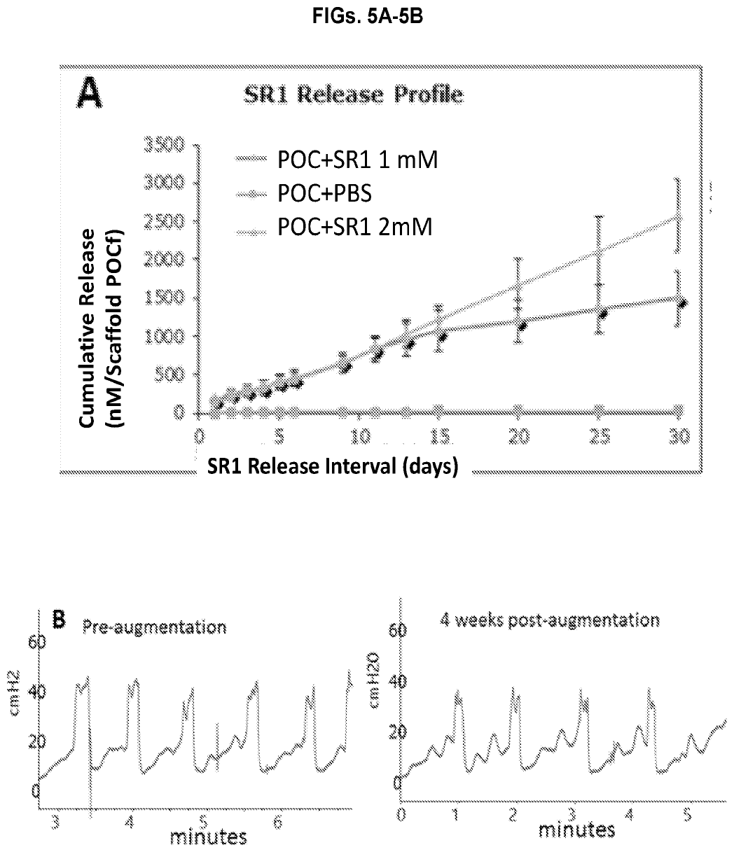

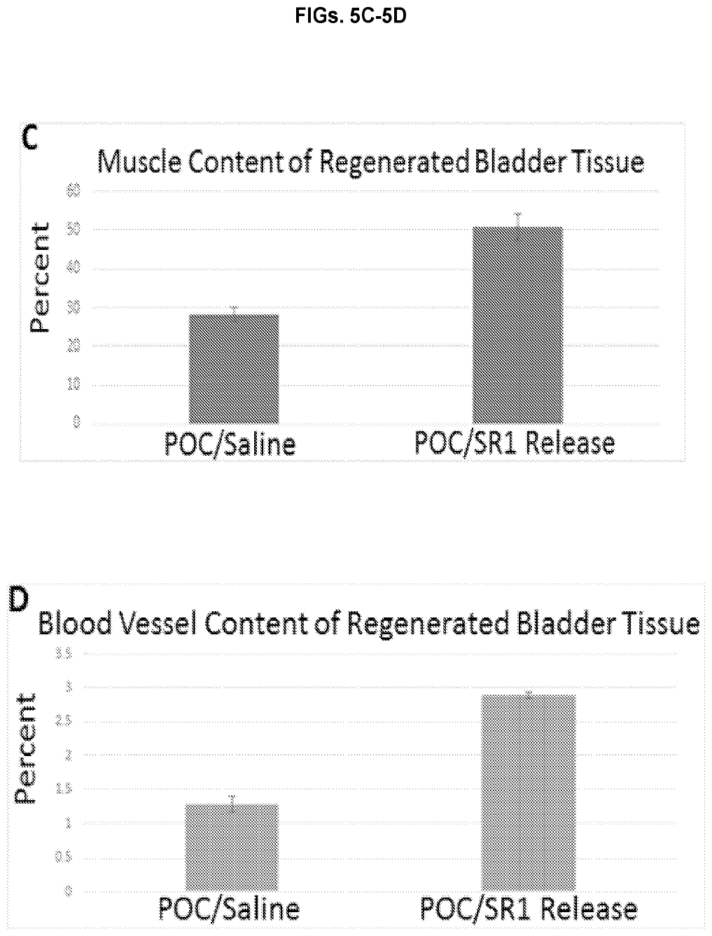

In some embodiments, administering comprises administering the AhR effector to bladder tissue of the subject. In some embodiments, administering the AhR effector to bladder tissue of the subject comprises administering an AhR effector coupled to a scaffold or released from an electronic actuator. In some embodiments, the AhR effector coupled to a scaffold is instilled in the bladder of a subject. In some embodiments, an electronic actuator comprising an AhR effector is implanted in the bladder of a subject. As used herein, “scaffold” refers to a substance which modulates the release characteristics of the AhR effector. In some embodiments, the scaffold comprises an elastomer, e.g., a biodegradable elastomer. As used herein, “elastomer” refers to a natural or synthetic polymer having elastic properties. In some embodiments, the scaffold comprises the elastomer poly (1,8 octanediol-co-citrate)(POC) (FIG. 5). As used herein, “coupled” describes the condition of two compounds being associated, but not necessarily chemically bound to one another. For example, in some embodiments, an AhR effector, e.g., SR1 is coupled to POC. As used herein, “electronic actuator” refers to an apparatus configured to release a compound, e.g., an AhR effector, upon stimulation with an appropriate signal by means of an electronically controlled actuator. Exemplary electronic actuators comprise micro-electro-mechanical devices (MEMS), etc.

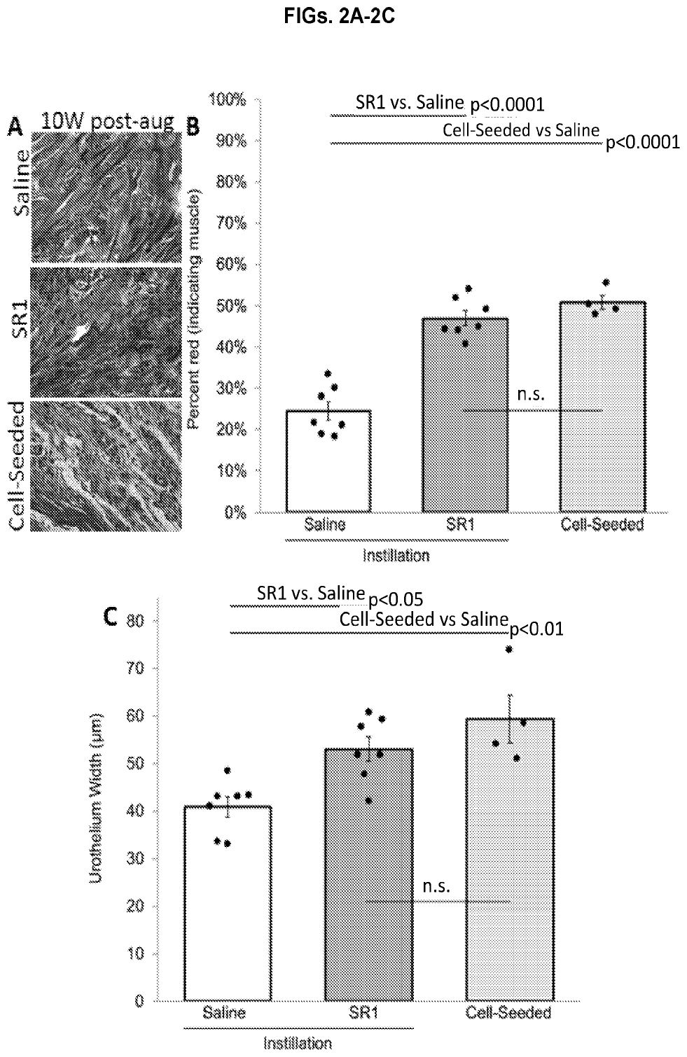

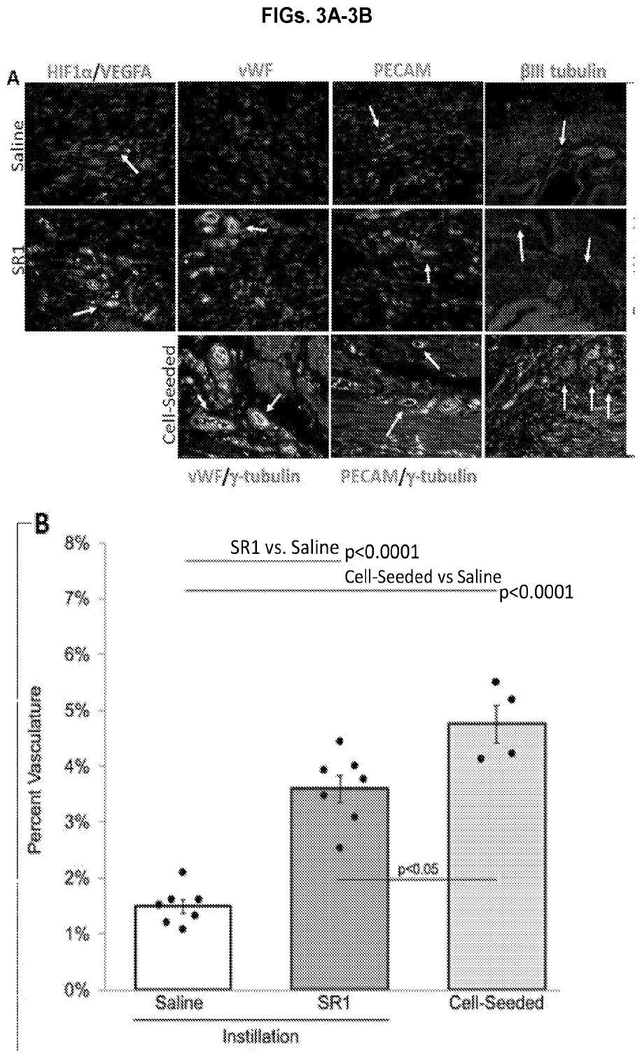

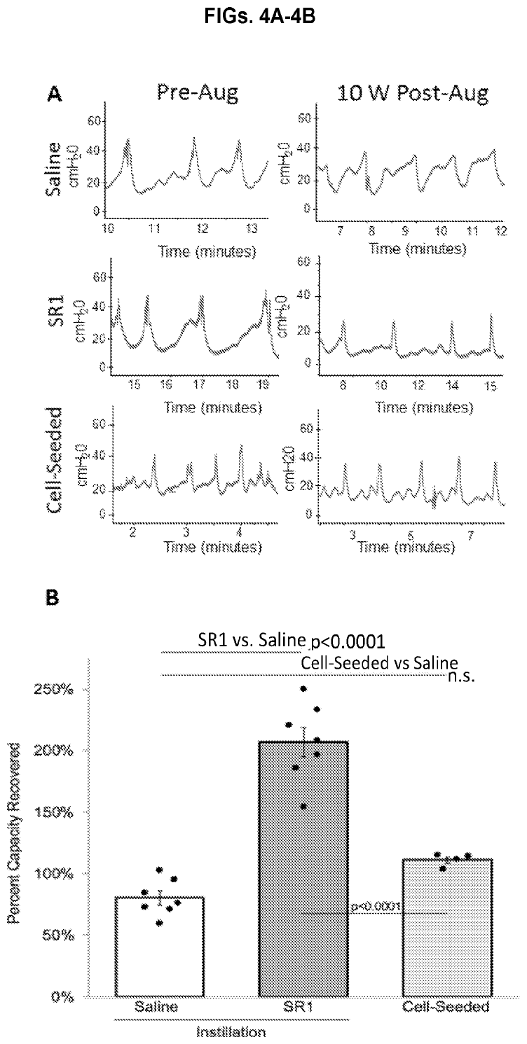

The inventors found that instillation of SR1 in rats that had undergone 60-70% bladder cystectomy caused bladder tissue regeneration. Remarkably, the regenerated bladder tissue consisted of a roughly 1:1 ratio of muscle to collagen (FIG. 2A [control] and FIG. 5B). Therefore, in some embodiments of the methods, the regenerated bladder tissue comprises a ratio of about 1:1 muscle to collagen. In some embodiments, the method reduces one or more symptoms of bladder dysfunction, for example, abnormal voiding, incontinence and subsequent infection, bladder distension, ureteral or renal dysfunction. In some embodiments, the method results in increased vascularization and peripheral nerve innervation in the regenerated bladder tissue. The inventor demonstrated that instillation of SR1 in grafted bladder tissue resulted in an abundance of peripheral nerve regeneration at the core and throughout the graft (FIG. 3). In some embodiments, the method results in recovery of at least about 100% of the subject's pre-surgery bladder capacity. Intriguingly, the inventor discovered that instillation of SR1 in bladder of rats which have undergone cystectomy lead to about a 200% percent recovery in bladder capacity, in comparison to original capacity, compared to saline-instilled animals (FIG. 4b).

In another aspect of the current disclosure, methods of promoting vascularization and peripheral nerve regeneration in surgically augmented urinary bladder tissue in a subject in need thereof are provided. In some embodiments, the methods comprise administering to the subject a pharmaceutical composition comprising an effective amount of an aryl hydrocarbon receptor (AhR) effector to promote vascularization and peripheral nerve regeneration in the surgically augmented bladder tissue. In some embodiments, the AhR effector is a compound with the following formula or a pharmaceutically acceptable salt thereof:

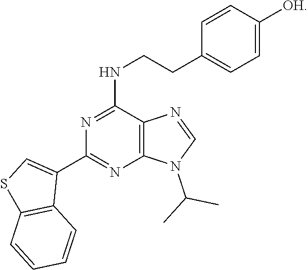 [SMILES: CC(C)n1cnc2c(NCCc3ccc(O)cc3)nc(-c3csc4ccccc34)nc21]

In some embodiments, administering comprises administering the AhR effector to bladder tissue of the subject. In some embodiments, administering the AhR effector to bladder tissue of the subject comprises administering an AhR effector coupled to a scaffold or released from an electronic actuator. In some embodiments, administering the AhR effector to bladder tissue of the subject comprises administering an AhR effector coupled to a scaffold or released from an electronic actuator. In some embodiments, the scaffold comprises the elastomer poly (1,8 octanediol-co-citrate) (POC) In some embodiments, the augmented bladder tissue comprises a ratio of about 1:1 muscle to collagen. In some embodiments, the method reduces one or more signs or symptoms of bladder dysfunction. In some embodiments, the method results in recovery of at least about 100% of the subject's pre-surgery bladder capacity.

In another aspect of the current disclosure, methods of increasing bladder capacity in a subject in need thereof are provided. In some embodiments, the methods comprise administering to the subject a pharmaceutical composition comprising an effective amount of an aryl hydrocarbon receptor (AhR) effector to increase bladder capacity. In some embodiments, the subject has undergone surgical bladder augmentation. In some embodiments, the AhR effector is a compound with the following formula or a pharmaceutically acceptable salt thereof:

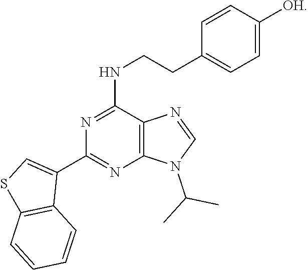 [SMILES: CC(C)n1cnc2c(NCCc3ccc(O)cc3)nc(-c3csc4ccccc34)nc21]

In some embodiments, administering comprises administering the AhR effector to bladder tissue of the subject. In some embodiments, administering the AhR effector to bladder tissue of the subject comprises administering an AhR effector coupled to a scaffold or released from an electronic actuator. In some embodiments, administering the AhR effector to bladder tissue of the subject comprises administering an AhR effector coupled to a scaffold or released from an electronic actuator. In some embodiments, the scaffold comprises the elastomer poly (1,8 octanediol-co-citrate) (POC). In some embodiments, the method reduces one or more signs or symptoms of bladder dysfunction.

In another aspect of the current disclosure, methods of inducing self-renewal and cellular differentiation of cells in a subject in need thereof are provided. In some embodiments, the methods comprise administering to the subject a pharmaceutical composition comprising an effective amount of an aryl hydrocarbon receptor (AhR) effector for inducing self-renewal and cellular differentiation of cells. In some embodiments, the cells comprise epithelial cells or endothelial cells. In some embodiments, the cells are selected from mammary, prostate, lung, blood vessel cells. In some embodiments, the AhR effector is a compound with the following formula or a pharmaceutically acceptable salt thereof:

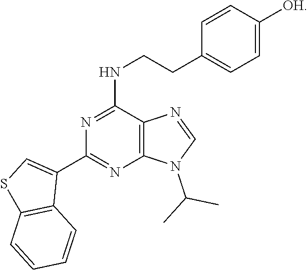 [SMILES: CC(C)n1cnc2c(NCCc3ccc(O)cc3)nc(-c3csc4ccccc34)nc21]

In another aspect of the current disclosure, compositions are provided. A composition comprising an aryl hydrocarbon receptor (AhR) effector coupled to a biodegradable elastomer. In some embodiments, the AhR effector is a compound with the following formula or a pharmaceutically acceptable salt thereof:

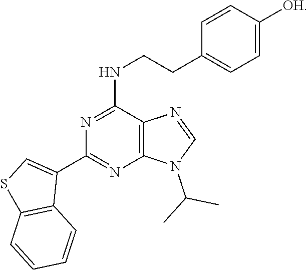 [SMILES: CC(C)n1cnc2c(NCCc3ccc(O)cc3)nc(-c3csc4ccccc34)nc21]

In some embodiments, the biodegradable elastomer comprises poly (1,8 octanediol-co-citrate).

The compounds employed in the methods disclosed herein may be administered as pharmaceutical compositions. Such compositions may take any physical form which is pharmaceutically acceptable; illustratively, they can be orally administered pharmaceutical compositions. Such pharmaceutical compositions contain an effective amount of a compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of aryl hydrocarbon receptor (AhR) may be administered as a single compound or in combination with another compound inhibits the biological activity of AhR or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of regenerating bladder tissue. By way of example, a treated subject may have increased bladder capacity, bladder control, increased innervation of the bladder tissue.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating regenerating urinary bladder tissue. By way of example but not by way of limitation, in some embodiments an effective amount is sufficient to result in one or more of increased bladder regeneration, increased bladder capacity, or increased innervation or muscular growth of the regenerated bladder tissue as compared to the subject before treatment, or compared to an appropriately matched, untreated control.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject: its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of regeneration of bladder tissue. A subject in need thereof may include a subject who has, for example, undergone bladder cystectomy or radical cystectomy, radiation treatment for urological cancers, or who suffers from interstitial cystitis (IC), spina bifida (SB) or trauma that necessitates regeneration of bladder tissue either from the primary condition or due to removal of native bladder tissue.

The term “subject” may be used interchangeably with the terms “individual” and “patient” and includes human and non-human mammalian subjects.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides, (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization”, as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

### Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —RC(O)N(R²)—, —R¹C(O)N(R²) R³—, —C(O)N R²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S”, depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### EXAMPLES

The following Examples are illustrative and should not be interpreted to limit the scope of the claimed subject matter.

### Example 1—Small Molecule Directed Urinary Bladder Tissue Regeneration

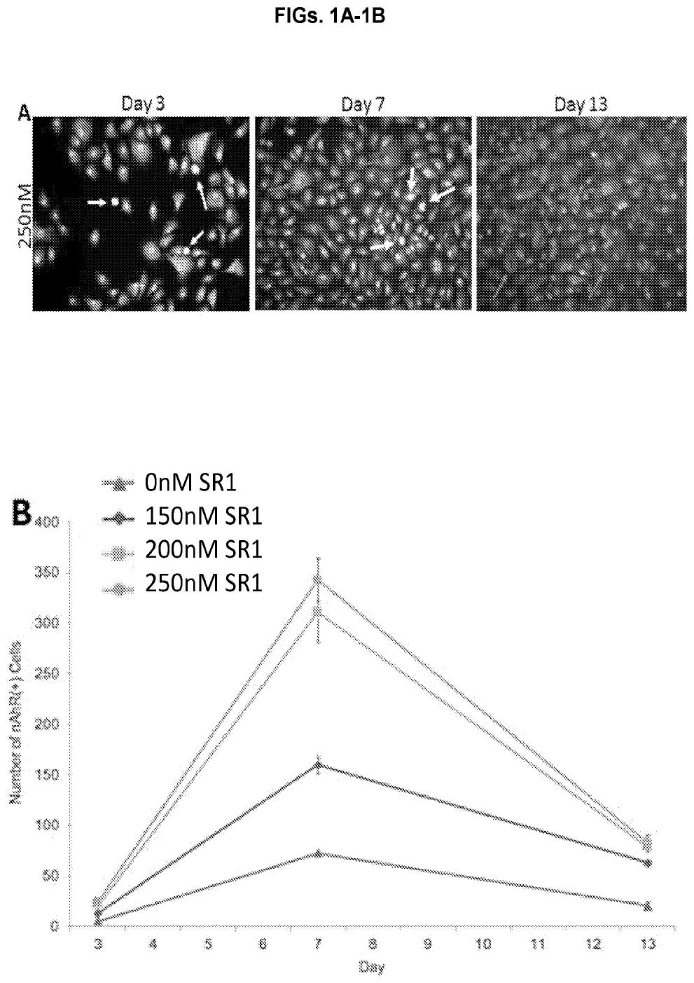

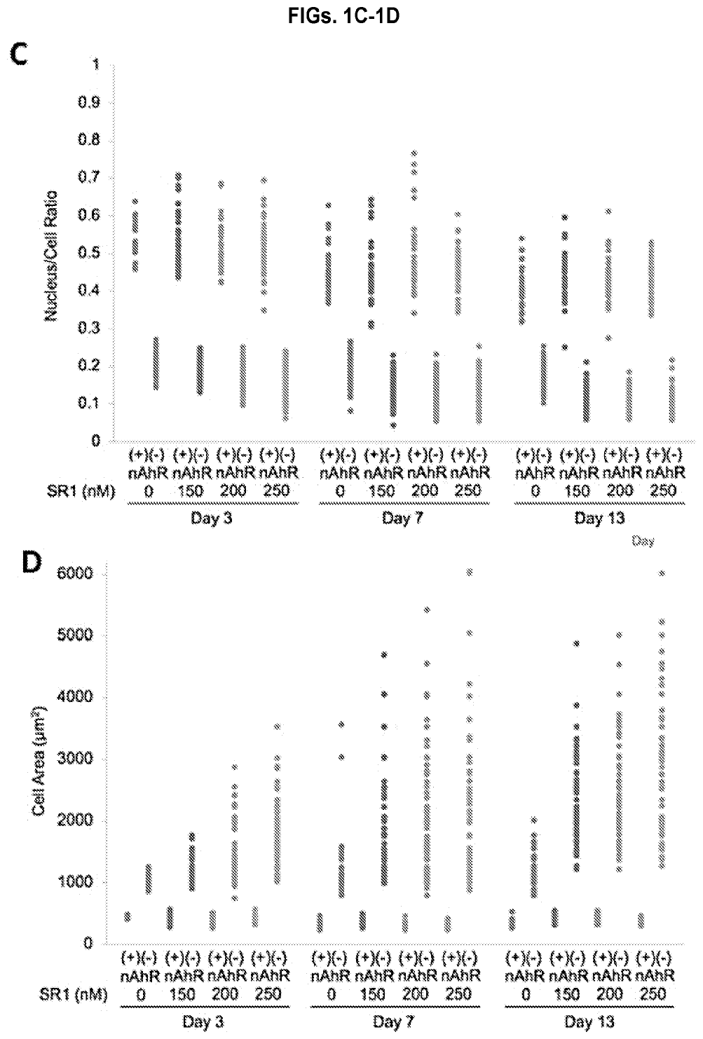

SR1 Treatment of UCs in vitro Equivalent numbers of primary human UCs were treated once with SR1 (150 nM, 200 nM, 250 nM, or untreated) and allowed to grow in culture for 13 days. An anti-AhR antibody (green, FIG. 3A) and mitotic proliferation/nuclear stain Ki-67 (blue) were used to co-stain UCs. nAhR+/Ki-67+UCs co-stained yellow and demonstrated a rounded shape (FIG. 1A, Days 3 and 7 white arrows). nAhR+/Ki-67+UCs were manually counted. Data demonstrate that the increasing concentration of SR1 resulted in increased numbers of umbrella-like cells suggesting terminally differentiated UCs (red arrows; FIG. 3A). Quantified data demonstrate an approximate 6× expansion of nAhR+/Ki-67+UCs at D7 (FIG. 1B) compared to untreated. An assortment of organ specific stem cells initially maintain a high nucleus/cytoplasm ratio which gradually decreases as the cell differentiates. FIG. 1C data demonstrate that nAhR+UCs can maintain a high nucleus/cytoplasm ratio and small cell area (FIG. 1D) indicative of primitive cells while nAhR−UCs differentiate into larger, mature cells (i.e., umbrella cells; FIG. 1A Day 13 and FIG. 1D) which is accentuated by SR1 treatment. Overall, data suggests that SR1 can simultaneously stimulate primitive UC self-renewal and differentiation analogous to hematopoietic stem cells previously reported.

SR1 Induces Bladder Tissue Regeneration in vivo—In the following in vivo studies, nude rats underwent an approximate 60-70% bladder cystectomy and were augmented with a POC scaffold and then: 1) instilled with saline (Saline); 2) instilled with 300 nM SR1 (SR1:1×/week for 2 weeks); or 3) seeded with human MSC/CD34+HSCs (Cell-Seeded). Animals were sacrificed 10 weeks post-augmentation. The inventors have developed, characterized, and successfully utilized CBBs (POC) in bladder regenerative settings over the last 10 years. The inventors' established human MSC/CD34+HSC seeded POC scaffold bladder augmentation model was utilized in this study to directly compare specific regenerative bladder biological metrics against SR1 treated animals to determine whether SR1 could be used in lieu of MSCs and CD34+HSCs for bladder tissue regeneration.

### Regenerated Bladder Tissue Muscle Content and Urothelium Width

Muscle to collagen ratios are indicative of overall bladder tissue regeneration. An established Trichrome staining/muscle quantification procedure revealed that regenerated bladder tissue of Saline instilled animals was mostly comprised of collagen compared to SR1 and Cell Seeded groups (FIG. 2A). Tissue quantitative morphometrics revealed that regenerated bladder tissue was ˜50% muscle in content at 10 weeks post-augmentation in SR1 and Cell-Seeded animals (FIG. 5B). Normal bladder tissue has an approximate 1:1 to muscle to collagen. There was no statistical difference with regards to muscle regeneration between the two groups. In vivo data demonstrate that the SR1 instilled animals recovered barrier attributes including urothelium that was properly spatially oriented and functional. Functionality was based upon observations that animals did not show any negative outward clinical symptoms of poor urothelial barrier function. This included abnormal voiding, incontinence and subsequent infection, bladder distension, ureteral or renal dysfunction, and normal urodynamics. Urothelium width (thickness; FIG. 2C) measurements were made from the basal layer of the urothelium to its apical layer from multiple aspects of the regenerated bladder tissue (see FIG. 2 Legend for numerical data). Rats have a urothelium width that ranges from 50-70 μm. Data provides further evidence that SR1 can be used to induce native bladder tissue growth.

### Bladder Tissue Vascularization and Peripheral Nerve Regeneration In Vivo

Bladder instillation of SR1 promoted vascularization of augmented bladders along the periphery and core of grafts. IF tissue staining with HIF1α (blue arrow for SR1 treated group; VEGFA, vWF and PECAM (white arrows, FIG. 3A, columns I-III) demonstrated abundant bladder tissue vascularization. As previously stated, AhR/ARNT driven transcription can modulate the up regulation of multiple pro-angiogenic genes including VEGFA and HIF1α. IF data from SR1 treated animals corroborate this finding in contrast to Saline instilled animals. Blood vessels in the Cell-Seeded groups co-stained with antibodies against vWF/Y-tubulin (a human specific marker) and PECAM/Y-tubulin demonstrated bladder tissue vascularization derived from cells of human origin (columns II and III, bottom). IF BIII tubulin staining (column IV; white arrows) demonstrated a lack of peripheral nerve regeneration in the Saline group in complete contrast to the SR1 instilled group and Cell-Seeded groups where there was an abundance of peripheral nerve regeneration at the core and throughout the graft. Vascular quantification of regenerated bladder tissue (FIG. 3B) demonstrated that Cell-Seeded grafts appear to provide a slight advantage with regards to percent tissue vascularization compared to the SR1 group. The level of tissue vascularization may be increased by changing the concentration of SR1 instilled and/or the dosing regimen.

Rat Bladder Urodynamic Studies (UDS) Rat bladders were filled with saline until a bladder contraction occurred (pre- and 10 weeks post-augmentation). Bladder pressures rose during the filling until bladder contractions occurred at intravesical pressures of 25-40 cm H2O. Saline instilled animals led to a slight decrease in voiding pressures and decreased bladder compliance (FIG. 4A). SR1 instilled animals led to a decrease in intravesical pressures during voiding from 40 to 25 cm H2O (pre-augmentation to post-augmentation, respectively) with increased bladder compliance. Cell-Seeded constructs maintained pre-aug intravesical pressures. With regards to leak point pressures (LPP); Saline, SR1 and Cell-Seeded groups (pre/post aug) were: 39.49±1.35/46.48±2.42; 39.66±0.81/37.1±1.47; 38.6±1.41/35.5±2.40 cm H2O, respectively. Compliance values for the same groups were: 68.77±2.49/39.3±8.55; 69.57±1.95/72.38±3.13; 46.23±12.9/89.59±1.32 percent, respectively. Normal LPP for rat is ˜40 cm H2O and compliant bladders are defined at >60%. Percent bladder capacity at 10 weeks was also measured (FIG. 4B). Data demonstrate the SR1 instilled animals were able to recover approximately 200% bladder capacity (see FIG. 4B legend for data regarding Percent Capacity Recovered of all groups). Data demonstrate that SR1 can be used instead of Cell-Seeded scaffolds.

### Example 2—SR1-Releasing Poly 1,8 Octane Diol Co-Citrate (POC) Scaffold Study

SR1 was coupled to POC (poly 1,8 octane diol co-citrate) scaffolds and its in vitro release profile was measured by spectroscopy, based upon the aromatic ring structures contained within SR1 (FIG. 5A). Data demonstrate a gradual release of SR1 at either 1 or 2 mM starting concentrations over 30 days compared to POC scaffolds in saline. For pilot in vivo studies, rats underwent an ˜70% cystectomy followed by augmentation with POC or POC/SR1 (1 mM SR1). POC animals were instilled with saline once at the beginning of the study and all animals were euthanized at 4 weeks post-augmentation. Pre- and post-augmentation urodynamics tracings for POC/SRT (FIG. 5B) and POC/Saline (data not shown) rats animals demonstrate an intravesical pressure of 40 cmH2O with normal filling/voiding patterns. Quantitative morphometrics of regenerated bladder tissue muscle demonstrated 28.13±1.69% and 50.91±3.34% (FIG. 5C) and vascular content was 1.29±0.11% and 2.89±0.03% (FIG. 5D) for POC/Saline and POC/SR1 scaffolds, respectively. POC/SR1 data demonstrate a regenerative advantage over POC/Saline scaffolds. Therefore, the inventor concludes that coupling of SR1 to a scaffold, e.g., a solid support, is an effective means of delivery of SR1 to subjects in need thereof.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for regenerating bladder tissue in a subject in need thereof, the method comprising administering to the subject a pharmaceutical composition comprising an effective amount of an aryl hydrocarbon receptor (AhR) effector for regenerating bladder tissue in the subject.

2. The method of claim 1, wherein the AhR effector is a compound with the following formula or a pharmaceutically acceptable salt thereof:

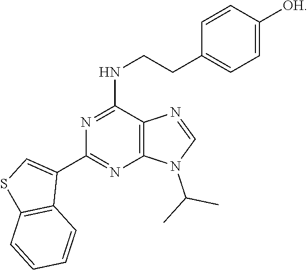 [SMILES: CC(C)n1cnc2c(NCCc3ccc(O)cc3)nc(-c3csc4ccccc34)nc21]

3. The method of claim 1 or 2, wherein administering comprises administering the effector to bladder tissue of the subject.

4. The method of claim 3, wherein administering the AhR effector to bladder tissue of the subject comprises administering the compound of claim 2 coupled to a scaffold or released from an electronic actuator.

5. The method of claim 4, wherein the scaffold comprises poly (1,8 octanediol-co-citrate) (POC).

6. The method of any of the preceding claims, wherein the regenerated bladder tissue comprises a ratio of about 1:1 muscle to collagen.

7. The method of any of the preceding claims, wherein the method reduces one or more symptoms of bladder dysfunction.

8. The method of any of the preceding claims, wherein the method results in increased vascularization and peripheral nerve innervation in the regenerated bladder tissue.

9. The method of any of the preceding claims, wherein the method results in recovery of at least about 100% of the subject's pre-surgery bladder capacity.

10. A method of promoting vascularization and peripheral nerve regeneration in surgically augmented urinary bladder tissue in a subject in need thereof, the method comprising administering to the subject a pharmaceutical composition comprising an effective amount of an aryl hydrocarbon receptor (AhR) effector to promote vascularization and peripheral nerve regeneration in the surgically augmented bladder tissue.

11. The method of claim 10, wherein the AhR effector is a compound with the following formula or a pharmaceutically acceptable salt thereof.

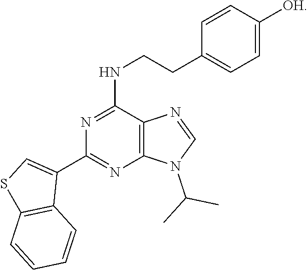 [SMILES: CC(C)n1cnc2c(NCCc3ccc(O)cc3)nc(-c3csc4ccccc34)nc21]

12. The method of claim 10 or 11, wherein administering comprises administering the AhR effector to bladder tissue of the subject.

13. The method of claim 12, wherein administering the AhR effector to bladder tissue of the subject comprises administering the compound of claim 11 coupled to a scaffold or released from an electronic actuator.

14. The method of claim 13, wherein the scaffold comprises poly (1,8 octanediol-co-citrate).

15. The method of any of claims 11-14, wherein the augmented bladder tissue comprises a ratio of about 1:1 muscle to collagen.

16. The method of any of claims 11-15, wherein the method reduces one or more signs or symptoms of bladder dysfunction.

17. The method of any of claims 11-16, wherein the method results in recovery of at least about 100% of the subject's pre-surgery bladder capacity.

18. A method of increasing bladder capacity in a subject in need thereof, the method comprising administering to the subject a pharmaceutical composition comprising an effective amount of an aryl hydrocarbon receptor (AhR) effector to increase bladder capacity.

19. The method of claim 18, wherein the subject has undergone surgical bladder augmentation.

20. The method of claim 18 or 19, wherein the AhR effector is a compound with the following formula or a pharmaceutically acceptable salt thereof:

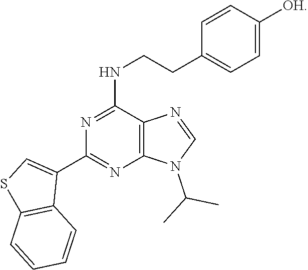 [SMILES: CC(C)n1cnc2c(NCCc3ccc(O)cc3)nc(-c3csc4ccccc34)nc21]

21. The method of any of claims 18-20, wherein administering comprises administering the AhR effector to bladder tissue of the subject.

22. The method of claim 21, wherein administering the AhR effector to bladder tissue of the subject comprises administering the compound of claim 20 coupled to a scaffold or released from an electronic actuator.

23. The method of claim 22, wherein the scaffold comprises poly (1,8 octanediol-co-citrate).

24. The method of any of claims 18-23, wherein the method reduces one or more signs or symptoms of bladder dysfunction.

25. A composition comprising an aryl hydrocarbon receptor (AhR) effector coupled to a biodegradable elastomer.

26. The composition of claim 25, wherein the AhR effector is a compound with the following formula or a pharmaceutically acceptable salt thereof.

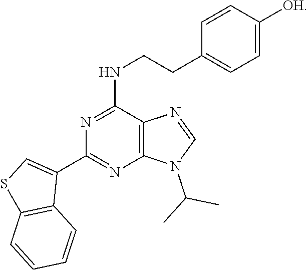 [SMILES: CC(C)n1cnc2c(NCCc3ccc(O)cc3)nc(-c3csc4ccccc34)nc21]

27. The composition of claim 25 or 26, wherein the biodegradable elastomer comprises poly (1,8 octanediol-co-citrate).

28. A method of inducing self-renewal and cellular differentiation of cells in a subject in need thereof, the method comprising administering to the subject a pharmaceutical composition comprising an effective amount of an aryl hydrocarbon receptor (AhR) effector for inducing self-renewal and cellular differentiation of cells.

29. The method of claim 28, wherein the cells comprise epithelial cells or endothelial cells.

30. The method of claim 28 or 29, wherein the AhR effector is a compound with the following formula or a pharmaceutically acceptable salt thereof: 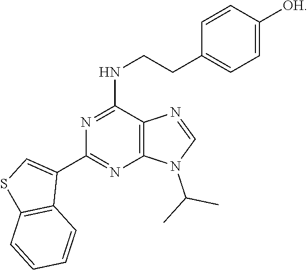 [SMILES: CC(C)n1cnc2c(NCCc3ccc(O)cc3)nc(-c3csc4ccccc34)nc21]
